(19) United States
(12) Patent Application Publication
(10) Pub. No.: US 2015/0034089 A1
(43) Pub. Date: Feb. 5, 2015

(54) SPEAKING VALVE SYSTEM WITH CUFF DEFLATION

(71) Applicant: Covidien LP, Mansfield, MA (US)

(72) Inventors: Kieran Donlon, Co. Westmeath (IE); Declan Kiernan, Co. Longford (IS); Colette Breheny, Co. Galway (IE); Phillip Gillen, Co. Longford (IE); Alan Finneran, Co. Offaly (IE)

(73) Assignee: Covidien LP, Mansfield, MA (US)

(21) Appl. No.: 13/958,333

(22) Filed: Aug. 2, 2013

(51) Int. Cl. A61M 16/04; A61M 16/00; A61M 16/20

(52) U.S. Cl. CPC A61M 16/0468; A61M 16/044; A61M 16/208; A61M 16/0463; A61M 16/0075
USPC 128/207.15

(57) ABSTRACT

A tracheal tube system includes a conduit and an inflatable cuff disposed on the conduit that seals a patient's airway. The tracheal tube system also includes an inflation line disposed along a portion of a length of the conduit and terminating in an opening within the inflatable cuff. The tracheal tube system further includes a speaking valve disposed at a proximal end of the conduit and in fluid communication with the inflatable cuff. The speaking valve is fluidly coupled to the inflatable cuff via a one-way valve such that fluid from the cuff flows into the speaking valve when the one-way valve is open.

### BACKGROUND

The present disclosure relates generally to the field of medical devices, and more particularly, to airway devices, such as speaking valve systems for use with tracheal tubes.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

A wide variety of situations exist in which artificial ventilation of a patient may be desired. For short-term ventilation or during certain surgical procedures, endotracheal tubes may be inserted through the mouth to provide oxygen and other gasses to a patient. For certain applications, particularly when longer-term intubation is anticipated, tracheostomy tubes may be preferred. Tracheostomy tubes are typically inserted through an incision made in the neck of the patient and into the trachea. A resulting stoma is formed between the tracheal rings below the vocal chords. The tracheostomy tube is then inserted through the opening.

Such tubes may include an inner cannula and an outer cannula, where the inner cannula may be disposed inside the outer cannula and used as a conduit for liquids or gas or medicine incoming and outgoing into the patient's lungs. The inner cannula may be removed for cleaning and for disposal of secretions while leaving the outer cannula in place, thus maintaining a desired placement of the tracheostomy tube. Other tracheostomy tubes may use only a single cannula. An inflatable cuff may be additionally provided, for securing the tracheostomy tube to the patient airway and blocking fluid flow around the outer cannula, thus enabling the tracheal tube to serve as a sole artificial conduit into the airway. A connector is typically provided at an upper or proximal end where the tube exits the patient airway, suitable for coupling the ventilator with the inner cannula. A set of flanges or wings are disposed around the outer cannula and used to securely couple the tracheostomy tube to the patient's neck.

To provide the patient the ability to breathe and speak, a one-way valve may be disposed over an end of the tracheostomy tube or connector that is external to the patient. Once in place, the one-way valve generally permits airflow to travel in only one direction within the tracheostomy tube. When the patient inhales, the check valve opens to allow air into the lungs. However, when the patient exhales, the check valve closes to enable the exhalation air to exit via the mouth and/or nose to facilitate speaking and breathing. When the one-way valve is in use, it may be desired to maintain the cuff in a deflated condition, thus enabling flow of air around the outer cannula and outwardly towards the vocal chords.

### BRIEF DESCRIPTION

This disclosure provides a novel speaking valve system designed to respond to such needs. The speaking valve system may include various implementations useful in deflating a cuff when the speaking valve is in use. After deflation of the cuff during use of the speaking valve, air may exit from the lungs and flow around the tracheal tube outwardly towards the vocal chords, improving speech and enhancing patient safety. In one example, a patient may be intubated and a cuff may be inflated, and, when the patient desires to speak, the speaking valve may be positioned onto a proximal end of the tracheal tube for use as a one-way check valve.

Thus, in accordance with a first aspect, a tracheal tube system is provided. The tracheal tube system includes a conduit and an inflatable cuff disposed on the conduit that seals a patient's airway. The tracheal tube system also includes an inflation line disposed along a portion of a length of the conduit and terminating in an opening within the inflatable cuff. The tracheal tube system further includes a speaking valve disposed at a proximal end of the conduit and in fluid communication with the inflatable cuff. The speaking valve is fluidly coupled to the inflatable cuff via a one-way valve such that fluid from the cuff flows into the speaking valve when the one-way valve is open.

In accordance with another aspect, a speaking valve is provided. The speaking valve includes a first one-way valve that opens during inspiration and a second one-way valve fluidly coupled to an inflatable cuff on a tracheostomy tube. The second one-way valve does not allow air to enter the inflatable cuff.

Also disclosed herein is a tracheostomy kit including a speaking valve that may be coupled to a tracheostomy tube and including a one-way valve that only delivers air into a patient's airway, a first syringe including one or more features that provide a first fixed volume representative of a first cuff size, and a second syringe including one or more features that provide a second fixed volume representative of a second cuff size.

### BRIEF DESCRIPTION OF THE DRAWINGS

Various aspects of the disclosed techniques may become apparent upon reading the following detailed description and upon reference to the drawings in which:

FIG. 1 is a perspective view of an exemplary tracheal tube assembly including a speaking valve having cuff deflation means, in accordance with aspects of present techniques;

FIG. 2 illustrates a patient having a tracheostomy system with the speaking valve of FIG. 1, according to embodiments of the present techniques;

FIG. 3 is a side view of the exemplary speaking valve of FIG. 1 showing a body having a one-way port;

FIG. 4 is a cross sectional view of the exemplary speaking valve of FIG. 1 showing an open one-way valve.

FIG. 5 is a cross sectional view of the exemplary speaking valve of FIG. 1 showing closed one-way valves.

FIG. 6 is a cross sectional view of a port of the exemplary speaking valve of FIG. 1 inserted into a PVT of the tracheostomy system of FIG. 1.

FIG. 7 is a side view of the speaking valve of FIG. 1 showing hinged cap;

FIG. 8 is a side view of the speaking valve of FIG. 1 showing a two-ported adapter;

FIG. 9 is a perspective view of the tracheal tube assembly including a speaking valve and an inflation line having cuff deflation means, in accordance with aspects of present techniques;

FIG. 10 is a side view of the speaking valve of FIG. 2 showing a valve disposed on a portion of a one-way port and the inflation line;

FIG. 11 is a perspective view of the tracheal tube assembly including an inflation line having cuff deflation means, in accordance with aspects of present techniques;

FIG. 12 is a perspective view of the tracheal tube assembly including an inflation line having bellows, in accordance with aspects of present techniques;

FIG. 13 is a side view of a syringe, in accordance with aspects of present techniques; and

FIG. 14 is a perspective view of the tracheal tube assembly including the syringe of FIG. 13, in accordance with aspects of present techniques

FIG. 15 is a side view of a syringe pump including the syringe of FIG. 13, in accordance with aspects of the present techniques.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Speaking valves are devices that are placed on a proximal end of a tracheal tube and that allow patients to speak although a tracheostomy tube is in place. The tracheal tube may be cuffed or cuffless and may also include fenestrations. In operation, a valve, e.g., a flutter valve, opens during spontaneous inspiration of a patient, and air is drawn through the valve and tracheal tube into the patient's lungs. Upon exhalation, the valve closes, forcing inhaled air to exit from the lungs and flow around the tracheal tube outwardly towards the vocal cords, allowing the patient to speak. In the case of a cuffed tracheal tube, the cuff may be deflated to allow the air to exit through the patient's trachea before the speaking valve is placed on the tracheal tube. Consequently, the cuff may be inflated once the patient is done speaking and or the speaking valve is removed. This may prove to be cumbersome to the patient and a caregiver. In addition, this may cause inadvertent stress to the patient and the caregiver due to deflating and inflating the cuff before and after use of the speaking valve. For example, the patient or the caregiver may not properly deflate the cuff before placing the speaking valve on the tracheal tube or may over inflate the cuff after use of the speaking valve, and therefore, cause patient discomfort. In addition, during use of fenestrated tracheal tubes, secretion buildup may occlude the fenestrations, thus preventing inhaled air from exiting the patient's airway. Accordingly, the cuff may be deflated to allow the inhaled air to exit the patient's airway, enabling the patient to speak and breathe. There currently exists a need for an improved speaking valve system that may deflate the cuff during use of the speaking valve to improve speech and enhance patient safety.

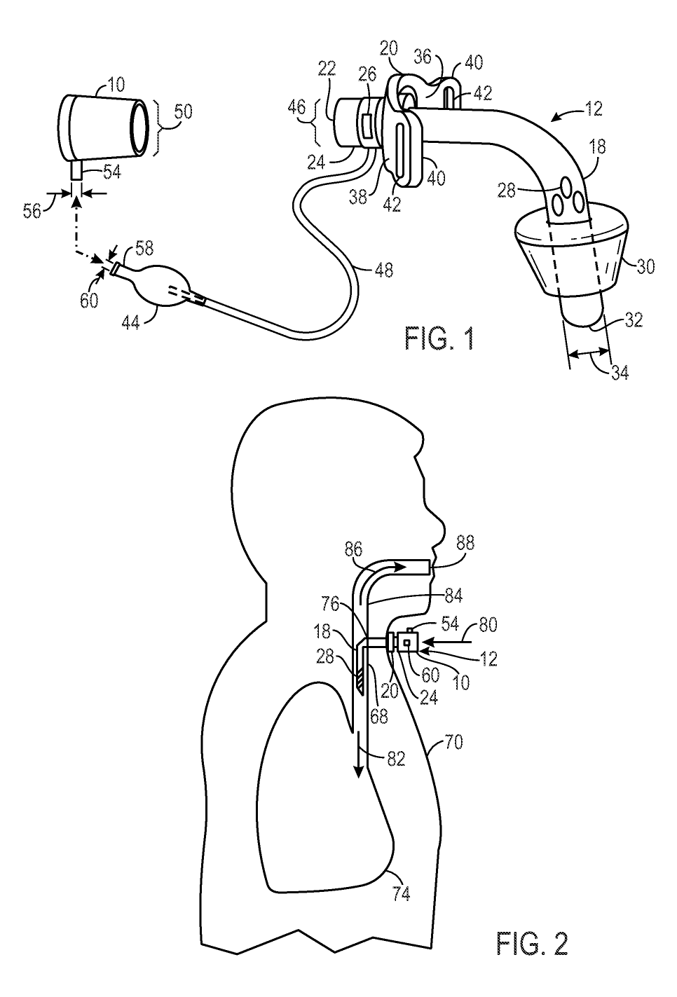

A tracheal tube system according to a preferred embodiment is illustrated in FIG. 1. In the depicted embodiment, a speaking or phonation valve 10 is included in a tracheal tube system 12, as illustrated. The application of the speaking valve 10 to a tracheostomy tube is apt, however, insomuch as such tubes tend to be worn for longer periods of time, and thus the speaking valve 10 may be used to facilitate speech even when the tracheal tube is inserted in a patient.

The tube system 12 may additionally include a removable and/or disposable inner cannula disposed inside of an outer cannula 18, useful in maintaining a clean ventilation circuit. However, it should be understood that some implementations may only include a single cannula, e.g., outer cannula 18.

The outer cannula 18 is illustrated extending both distally as well as proximally from a flange member 20. When used, the inner cannula may be introduced through an opening 22 of an end connector 24 and disposed inside of the outer cannula 18. During intubation, a tracheal tube assembly 26 including the inner cannula and outer cannula 18 is placed through an opening formed in the neck and trachea of a patient, and extending into the patient's airway. The tube assembly 26 embodiment illustrated in the figures includes a balloon cuff 28, although in practice a wide range of tube designs may be used, including tubes having no cuffs or tubes having multiple cuffs around the outer cannula 18. Further, the tubes may include fenestrations 30 above the balloon cuff 28 that allow air to flow outwardly through the patient's airway. In embodiments where the inner cannula is used, the inner cannula may also include fenestrations that align with the fenestrations 28 when the inner cannula is inserted into the outer cannula 18. As such, air flowing through the inner cannula may exit through the fenestrations and into the patient's airway.

In use, the balloon cuff 28 may be inflated so as to expand and contact the patient's airway. The outer cannula 18 in the illustrated embodiment may then form the sole conduit from which liquids or gases, including medications, may enter through the proximal opening 22 and exit through a distal opening 32. When the inner cannula is used, the inner cannula forms the gas flow interior passage. The outer cannula 18 has an outer dimension 34 allowing it to fit easily through an incision made in the neck and trachea of the patient. In practice, a range of such tubes may be provided to accommodate the different contours and sizes of patients and patient airways. Such tube families may include tubes designed for neonatal and pediatric patients as well as for adults. By way of example only, the outer dimension 34 of the outer cannula 18 may range from 4 mm to 16 mm.

In one embodiment, the outer cannula 18 extends from the flange member 20 along a lower face 36 and protrudes through an upper face 38 of the flange member 20. When in use, the face 36 will generally be positioned against the neck of a patient, with the cannula extending through an opening formed in the neck and trachea. A pair of side wings or flanges 40 extend laterally and serve to allow a strap or retaining member to hold the tube assembly in place on the patient. In the illustrated embodiment, apertures 42 are formed in each side flange member 20 to allow the passage of such a retaining device. In many applications, the flange member 20 may be taped or sutured in place as well.

The end connector 24 is formed in accordance with industry standards to permit and facilitate connection to ventilating equipment (not shown) and to the speaking valve 10. By way of example, standard outer diameters may be provided as indicated at reference numeral 46 that allow a mating connector piece to be secured on the connector shown. By way of example, a presently contemplated standard outer diameter (OD) 46 accommodates a 15 mm connector, although other sizes and connector styles may be used. In use, then, air or other gas may be supplied through the connector 24 and the outer cannula 18, and gases may be extracted from the patient. For example, the tube system 12 may be inserted into the patient's airways, and the balloon cuff 28 may then be inflated through an inflation conduit 48 fluidly coupled to an inflation lumen (not shown) extending along an inner wall of the outer cannula 18 and terminating in an opening within the balloon cuff 28. A Pilot Valve Tail (PVT) 44 may then indicate that air is in the balloon cuff 28, for sealing the patient's airway. Once the tracheal tube assembly 12 is positioned and secured, a ventilator may be coupled to the end connector 24.

When the patient wishes to speak, the ventilator may be removed and replaced with the speaking valve 10. The speaking valve 10 may include an inner diameter (ID) 50 sized to mate with the end connector 24. In use, the speaking valve 10 is disposed over the end connector 24 and used to provide for one-way air intake into the patient's airway, as described in more detail below with respect to FIG. 2. In one embodiment, the speaking valve 10 may include a port 54 having an outer diameter (OD) 56 sized and shaped to be fluidly coupled to the balloon cuff 28, e.g., via the inflation conduit 48 and PVT 44. For example, the port 54 may be inserted within a bore 58 extending from a proximal end of the PVT 44 and having an ID 60 slightly larger than the OD 56. In one embodiment, the coupling opens a valve that is at a terminus of the PVT 44 to allow air to flow out of the conduit 48. Alternatively, tubing having a suitable diameter and length may be used to indirectly connect the port 54 to the bore 58. The port 54 may include a one-way valve (not shown) that allows air to only enter the speaking valve 10. One-way valves may include check valves, ball valves, diaphragm valves, swing valves, check bell valves, etc. In use, during patient inhalation, air from the balloon cuff 28 enters the speaking valve 10 through the port 54 and the balloon cuff 28 is deflated. Because the port 54 includes a one-way valve, air does not re-enter the inflation conduit 48 to re-inflate the cuff 28. Accordingly, the cuff 28 remains deflated when coupled to the speaking valve 10. In another embodiment, the port 54 may be useful for fluidly coupling the speaking valve 10 to an air supply, such as an oxygen canister. For example, once the cuff 28 has been deflated, the port 54 may be removed from the bore 58 and connected to the oxygen canister. Accordingly, supplemental oxygen may be delivered to the patient during use of the speaking valve 10.

FIG. 2 shows the tracheostomy system 12 that has been inserted into a trachea 68 of a patient 70. The tracheostomy system 12 provides controlled access to the lungs 74 of the patient 70 via a stoma 76 on an anterior portion of the neck. As depicted, the system 12 provides a fluid pathway to the lungs 74. The flange 20 is disposed near the proximal end of the cannula 18 and rests on the anterior portion of the neck to provide stability to the system 12. At the proximal tip of the cannula 18, the connector 24 provides a connection point for attaching additional airway accessories to the system 12. Such an accessory may be the speaking valve 10, which enables the patient 70 to speak and breathe independently while the system 12 is disposed in the patient 70. As detailed below, the speaking valve 10 may include features for deflating the balloon cuff 28 when the patient desires to speak.

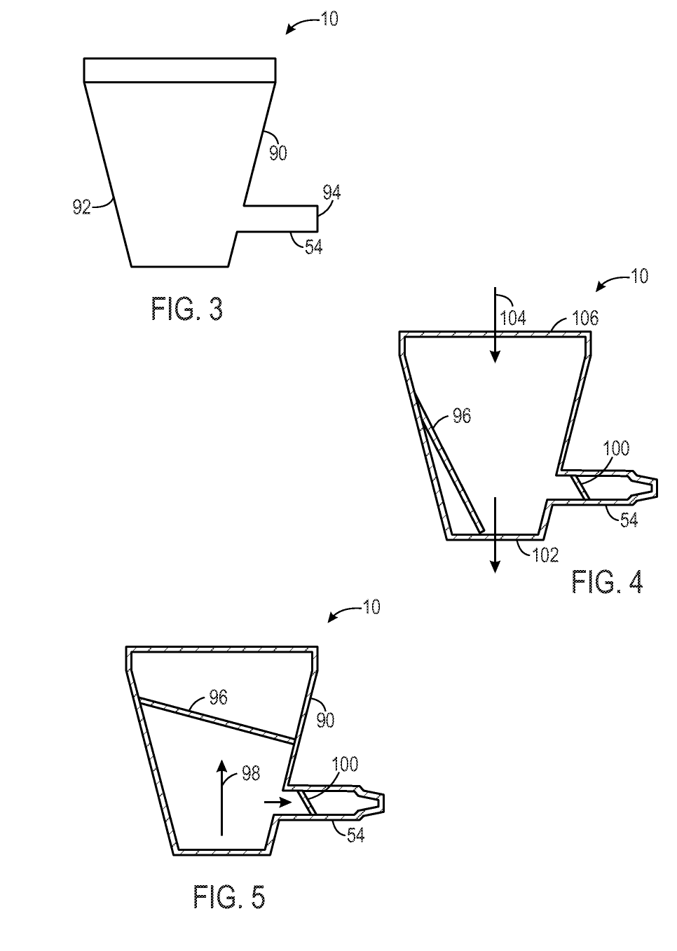

When the speaking valve 10 is used in conjunction with the system 12, the port 54 may be used to deflate the balloon cuff 28, as mentioned above. To facilitate speaking, the speaking valve 10 acts as a one-way check valve and allows only inhalation air, indicated by arrow 80, to travel through to the system 12 into the lungs 74. The inhalation air exits the distal end of the cannula 18 and enters the lungs 74, as indicated by arrow 82. When exhalation begins, the valve 10 may then block the air from exiting the patient 70 via the system 12, forcing the air around the system 12 to pass the larynx 84, as indicated by arrow 86. The larynx 84 houses vocal folds, which vibrate as the air (following arrow 86) flows past. Vibration of the vocal folds facilitates phonation. When the patient speaks, the exhalation air exits the patient 70 via mouth 88. Further details of the use of the port 54 to deflate the balloon cuff 28 are described with respect to FIG. 3.

Turning now to FIG. 3, a side view of an embodiment of a speaking valve 10 having the port 54. As discussed above, the speaking valve 10 may be in fluid communication with the balloon cuff 28 through the conduit 48. The port 54 extends outwardly from an outer surface 90 of a body 92 of the speaking valve 10. The port 54 may be any shape suitable for facilitating connection to the bore 58. For example, a terminal end 94 of the port 54 may be tapered so as to be easily inserted into the bore 58. In other embodiments, the terminal end 94 may include internal or external threads, or a combination thereof, such that fittings may be used to secure the port 54 to the bore 58 or tubing. When speech is desired, the speaking valve 10 may be coupled to the end connector 24 to provide for one-way check valve functionality as described previously. Advantageously, the balloon cuff 28 may be deflated, for example during patient inhalation (e.g., by inhaling the air inside the balloon cuff 28) and air flows through an annulus between the trachea 68 and the outer cannula 18 during exhalation. In other embodiments, as discussed above, an oxygen canister may be coupled to the port 54 to supply supplemental oxygen to the patient through the speaking valve 10 during use.

As discussed above, the speaking valve 10 includes a valve to allow air to only enter the system 12. FIG. 4 is cross sectional view of an embodiment of the speaking valve 10 having valves 96 and 100. The valves 96 and 100 open during inspiration. For example, when the patient inhales, the valve 96 moves downwardly towards a distal end 102, and thereby opening the valve 96. This allows air to enter and flow through the speaking valve 10 into the system 12, as indicated by arrow 104. Similarly, the valve 100 opens upon inhalation to allow air to flow into the speaking valve 10 through the port 54. In embodiments where the port 54 is fluidly coupled to the inflatable cuff 28, the air from within the cuff is drawn into the speaking valve 10 and the cuff is deflated.

To facilitate phonation, the speaking valve 10 may only allow air to enter the system 12. Accordingly, the valves 96 and 100 are closed during expiration. For example, the valve 96 may move upwardly towards a proximal end 106 to seal the speaking valve 10, as illustrated in FIG. 5. When the valve 96 seals the speaking valve 10 the inhaled air may not exit, as indicated by arrow 98, and thereby forcing the air to flow out the patient's airway. Concurrently, the valve 100 seals the port 54 and the inhaled air is directed towards the system 12 and into the patient's airway.

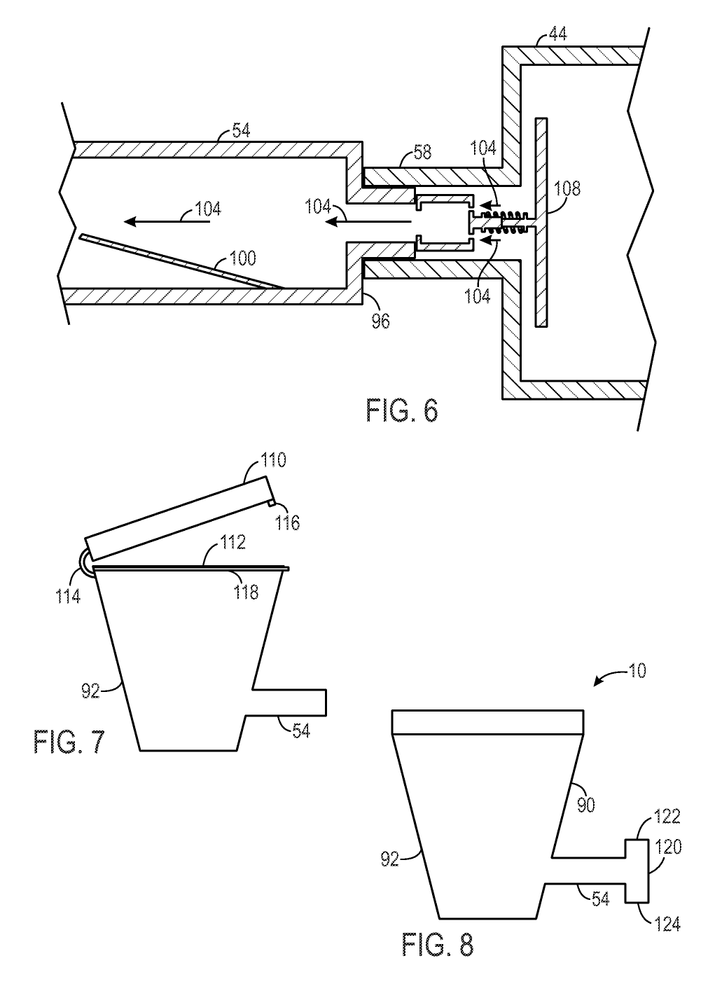

Turning now to FIG. 6, a cross sectional view of the speaking valve 10 coupled to the PVT 44 is illustrated. In the illustrated embodiment, the distal end 94 includes a male luer connector that is inserted into a corresponding female luer connector on the PVT 44. When the speaking valve 10 is coupled to the PVT 44, the male luer connector opens a valve 108 in the female luer connector by pushing the valve 108 inwardly and away from a proximal end of the PVT 44. This provides fluid communication between the port 54 and the inflation cuff 28 and air may flow into the port 54. For example, upon inhalation, the valve 100 opens and air, arrows 104, from the inflatable cuff 28 may flow into the speaking valve 10, as discussed above. As should be understood, decoupling of the port 54 and the PVT 44 may close the valve 108 and air may not flow out through the PVT 44.

The speaking valve 10 may also include features to control the amount of air flowing through the speaking valve 10 and into the lungs 74. For example, in one embodiment shown in FIG. 7, the speaking valve 10 may include a cap 110 on a proximal end 112 of the speaking valve 10. The cap 110 may be removed by the patient or the caregiver to allow additional air to enter the speaking valve 10 during inhalation. By removing the cap 110 the amount of air flowing into the lungs may increase by approximately 60% to 80% compared to the amount of air flowing into the lungs when the cap 110 is closed. This increase in air flow may allow the patient to speak more comfortably. It may also reduce the use of supplemental oxygen in certain patients during use of the speaking valve 10.

The cap 110 may be coupled to the body 92, for example, by using a hinge 114. The hinge 114 may be provided by molding or overmolding the hinge 114 externally to the cap 110 and the body 92 and used, for example, as the sole member connecting the cap 110 and the body 92. The hinge 114 may be formed from the same material as the speaking valve 10 of any other suitable durable and flexible material. In one embodiment, the hinge 114 may include a spring. The spring may force the cap 110 outwardly and away from the body 92 opening the proximal end 112 of the speaking valve 10. As such, the cap 110 may remain completely open for as long as the patient desires without obstructing any portion of the proximal end 112. The cap 110 may also include one or more protrusions 116 that snap onto, for example, a rim 118.

The speaking valve 10, as provided herein, may be manufactured from any suitable material, such as polymers, resins, composites, or a combination thereof. In one example, the speaking valve 10 and its components may be manufactured out of a material such as polyvinylchloride, a polyurethane, thermoplastic elastomers, a polycarbonate plastic, silicon, an acrylonitrile butadiene styrene (ABS), or a polyvinyl chloride (PVC), rubber, neoprene, or combination thereof. Likewise, the components of the tracheal tube assembly 12 may be manufactured of polyvinylchloride, polyurethane, thermoplastic elastomers, polycarbonate plastic, silicon, ABS, PVC, rubber, neoprene, or combination thereof.

As discussed above, when the speaking valve 10 is coupled to the system 12, the port 54 may be used to deflate the balloon cuff 28. Turning now to FIG. 8, the port 54 may include additional features to assist in deflating the balloon cuff 28. One such feature may include an adapter 120 disposed on the terminal end 96 of the port 54. The adapter 120 may include ports 122 and 124, forming a T-junction with the distal end 96. In one embodiment, the port 124 may be connected to the bore 58 and the port 122 may be connected to a vacuum line. Accordingly, the balloon cuff 28 may be in fluid communication with the vacuum line and the balloon cuff 28 may be quickly deflated upon applying a vacuum. This may be advantageous, for example, if the cuff was not properly deflated during inhalation as a result of shallow breathing or if tube fenestrations are blocked due to secretion build-up. In another embodiment, the port 122 may be coupled to a syringe such that the patient may control deflation and/or inflation of the balloon cuff 28 without the need for a vacuum pump. This may be useful outside of a non-clinical setting.

The adapter 120 may be configured to provide selective fluid communication between the port 54 and the bore 58. For example, the adapter 120 may include a two-way valve to direct the flow of air within the balloon cuff 28 to the port 54 during inhalation of the patients. Similarly, the two-way valve may direct the flow of air from the balloon cuff 28 to the port 122 for instantaneous deflation of the balloon cuff 28. The adapter 120 may be molded or overmolded onto the port 54. In other embodiments, the adapter 120 may be a separate structure and removably attached to the distal end 96. For example, the adapter 120 may include a protrusion that may be inserted into the distal end 96 and secured to the port 54 with a fitting.

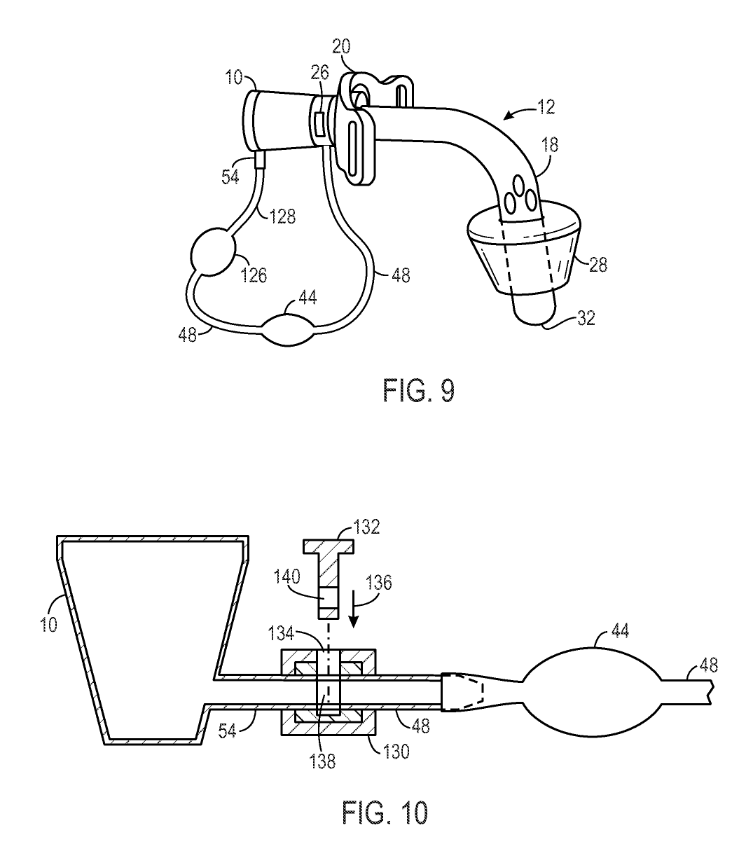

In a further embodiment, the speaking valve 10 may be attached to an inflation bellows. FIG. 9 illustrates the system 12 including the speaking valve 10 and an inflation bellows 126 disposed on inflation conduit 48 proximal to the PVT 44. The inflation bellows 126 may be coupled to the port 54 through conduit 128, however the inflation bellows 126 may also be directly attached to the distal end 96 of the port 54. In use, the inflation bellows 126 may be compressed to remove the air within the balloon cuff 28 and open the flutter valve within the port 54. Accordingly, the air from within the balloon cuff 28 may flow into the speaking valve 10, deflating the balloon cuff 28 and facilitate speech as discussed above. Similarly, once the patient has finished speaking, the inflation bellows 126 may be compressed and air may be delivered to the balloon cuff 28 and inflating the cuff.

In yet a further embodiment, the speaking valve 10 may be coupled to a deflation valve 130 disposed proximal to the PVT 44 at a proximal end of the conduit 48, as illustrated in FIG. 10. In the illustrated embodiment the valve 130 includes a pin 132 that may be inserted into an opening 134, as indicated by arrow 136. In one embodiment, the pin 132 blocks a passageway 138 of the valve 130, preventing air within the cuff to flow into the speaking valve 10. The balloon cuff 28 may be deflated by opening the deflation valve 130. In one embodiment, the deflation valve 130 may be opened by twisting the pin 132 so that a passageway 140 of the pin 132 is aligned with the passageway 138, releasing the air within the balloon cuff 28 into the port 54. Once the balloon cuff 28 has been deflated, the valve 130 may remain in an open position.

In another embodiment, the pin 132 may not include the passageway 140, and, as such the pin 132 may be completely removed from the opening 134 such that the air within the balloon cuff 28 may flow into the speaking valve 10 or flow out through the opening 134. In a further embodiment, the pin 132 may be partially pulled outwardly towards the opening 134, unblocking the passageway 138 and allowing air from within the balloon cuff 28 to flow into the speaking valve 10 during inhalation. As should be noted, once the balloon cuff 28 is re-inflated the deflation valve 130 is placed in a closed position by twisting the pin 132 such that the passageway 140 is not aligned with the passageway 138 to prevent undesired deflation of the balloon cuff 28.

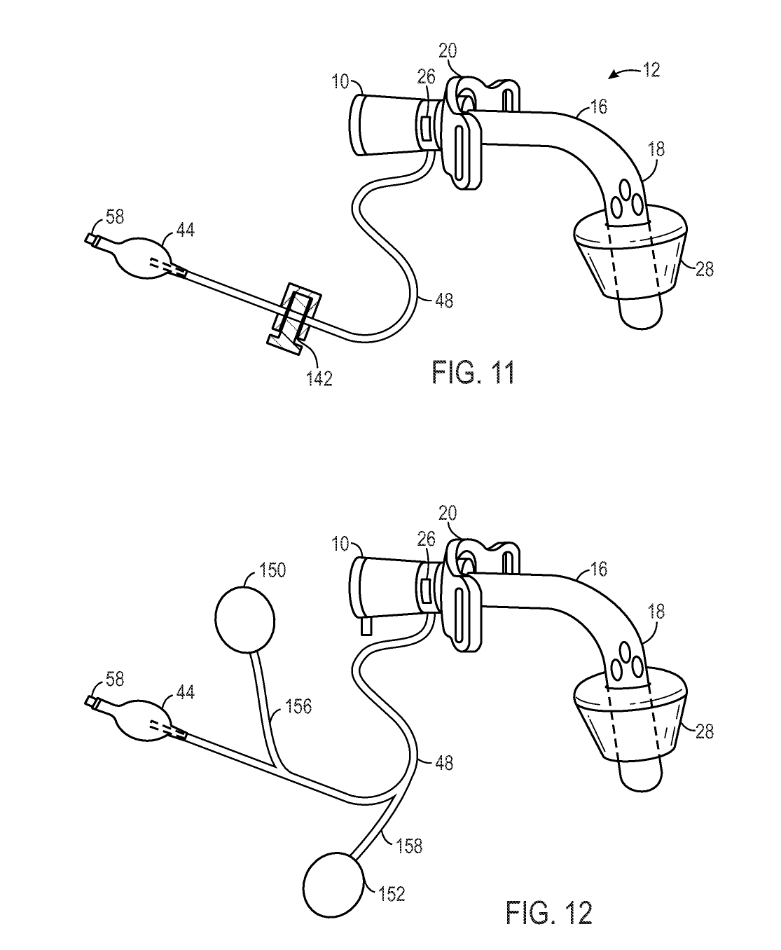

Although the speaking valve 10 may be used to deflate the balloon cuff 28 during inhalation, the system 12 may also include additional features for deflating the balloon cuff 28. FIG. 11 illustrates the system 12 including a separate cuff deflation feature. In the illustrated embodiment, the inflation conduit 48 includes a two-way valve 142 disposed distal to the PVT 44. In use, the two-way valve 142 may deflate the balloon cuff 28 by opening the valve 142 to allow air from the balloon cuff 28 to flow out through the PVT 44. Accordingly, air flow entering the inner cannula 14 from the speaking valve 10 may flow out the patient's airway enabling phonation, as discussed above. The valve 142 may remain opened or closed during use of the speaking valve 10. In certain embodiments, a suction source (e.g., vacuum, syringe) may be coupled to the PVT 44 through bore 58 to deflate the balloon cuff 28. The balloon cuff 28 may be re-inflated through the PVT 44, with the valve 142 in the open position, once the patient has finished speaking. Upon inflation of the balloon cuff 28, the valve 142 may be closed, sealing the conduit 48, to maintain the balloon cuff 28 in an inflated state. It should be noted that the valve 142 may also be used as a relief valve for the balloon cuff 28. For example, during use, the balloon cuff 28 may build up pressure due to the flow of gasses in the system 12 and the patient's airway, causing the balloon cuff 28 to become overinflated. The valve 142 may be briefly opened to relieve the pressure build-up in the balloon cuff 28.

In another embodiment, the inflation conduit 48 may include a fluid source and a fluid reservoir that may move air in and out of the balloon cuff 28. FIG. 12 illustrates the system 12 including bellows 150 and 152. The bellows 150 and 152 are disposed on the ends of conduits 156 and 158, respectively, disposed distal to the PVT 44 and extending from, and in fluid communication with, inflation conduit 48. To facilitate phonation, as discussed above, the speaking valve 10 is coupled to the outer connector 24, the balloon cuff 28 is deflated, and air enters the speaking valve 10. In the illustrated embodiment, the balloon cuff 28 may be deflated by compressing the bellows 150 causing air to be suctioned out of the balloon cuff 28. The air from the deflated balloon cuff 28 may flow into the bellows 152 through conduit 158 and phonation is facilitated as described above. Once phonation is complete, the bellows 152 may be compressed to return the air back to the balloon cuff 28, inflating the cuff. Because the balloon cuff 28 may be deflated and inflated with the bellows 150 and 152, respectively, it may not be necessary to used additional equipment (e.g., vacuum, syringe, air supply, etc.) to deflate or inflate the balloon cuff 28. It should be noted that the balloon cuff 28 may also be deflated and/or inflated through PVT 44. For example, in embodiments where instantaneous deflation is needed, a vacuum may be coupled to the PVT 44 and air within the balloon cuff 28 may be suctioned out. The PVT 44 may also be used to add additional air to the balloon cuff 28. For example, the air within the balloon cuff 28 may occasionally need to be adjusted to maintain a proper seal of the patient's airway, accordingly, the air supply may be coupled to the bore 58 to deliver air to the balloon cuff 28.

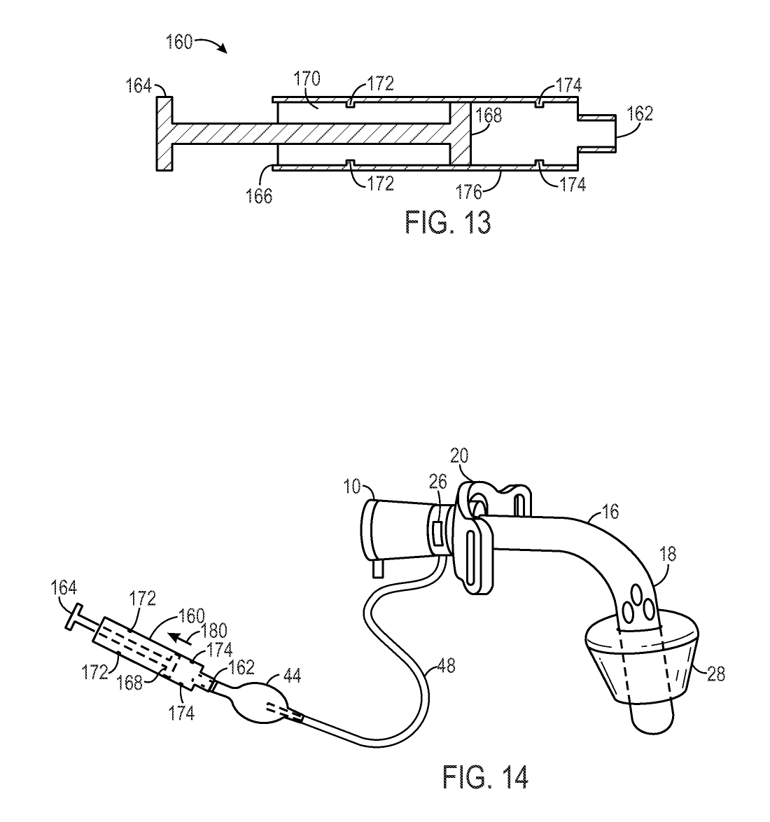

The system 12 may also be coupled to an inflation/deflation syringe. FIG. 13 illustrates a syringe 160 that may be used with the system 12 to inflate and deflate the balloon cuff 28. The syringe 160 includes a distal tip 162 for coupling to the system 12 and a plunger 164 inserted into a proximal end 166 for moving air in and out of the syringe 160. A distal end 168 of the plunger may have a diameter equal to an ID of the syringe 160 such that the distal end 160 of the plunger abuts an inner wall 170 and provides an airtight seal. The inner wall 170 may include stopping features 172 and 174 that restrict movement of the plunger 164 within the syringe 160. For example, the stopping features 172 and 174 may include notches, recesses, protrusions, or a combination thereof that may stop the plunger 164 from moving past the stopping features 172 and 174. The stopping features 172 and 174 may provide an alignment mechanism, for example, by preventing the patient or the caregiver from over deflating or overinflating the balloon cuff 28, as will be described in detail below. In certain embodiment, an exterior surface 176 of the syringe 160 may include markings indicative of a cuff deflation/inflation status. For example, the surface 176 may include markings such as DEFLATE, SPEAK, OPEN, or any other suitable marking indicative of cuff deflation at stopping feature 172. Correspondingly, stopping feature 174 may include markings such as INFLATE, NO SPEECH, CLOSE, or any other suitable markings indicative of cuff inflation. Advantageously, these markings may minimize the risk of inadvertently over deflating or overinflating the balloon cuff 28.

In other embodiments, the markings may indicate a volume of air withdrawn or delivered to the balloon cuff 28. For example, the surface 176 may have a volume guideline marking, such as between 15 cc and 30 cc, at stopping feature 172 to indicate when a desired amount of air has been withdrawn from the balloon cuff 28, and therefore deflating the cuff. Similarly, the surface 176 may have the volume guideline marking of 0 cc at stopping feature 174 to indicate that the volume of withdrawn air has been delivered to the balloon cuff 28. The volume guidelines above are only exemplary and may vary dependent on the size of the cuff used in the system 12. Accordingly, in certain embodiments, the system 12 may include a dedicated syringe with volume guideline markings for the specific cuff used in the system 12, for example, as part of a kit.

As discussed above, the syringe 160 may be coupled to the system 12 to deflate and inflate the balloon cuff 28. Turning now to FIG. 14, the system 12 including the syringe 160 coupled to the PVT 44 is illustrated. In certain embodiments, the syringe 160 may be molded or overmolded onto the PVT 44 to form a single piece. In other embodiments, the syringe 160 may be removable and only attached to the PVT 44 when needed for inflating or deflating the balloon cuff 28. Because the patient may use the system 12 outside a medical setting, the inflation conduit 48 may have a longer length than generally used to facilitate use of the syringe 160. For example, in one embodiment, the inflation conduit 48 may be long enough for the patient to place the syringe 160 in pocket on their clothing when coupled to the PVT 44. Although any suitable length may be used, in certain embodiments, the inflation conduit 48 may have a length of approximately between 10 inches to 60 inches or more.

The inflation conduit 48 may also include features to reduce tangling or kinking that may result from the extended length and be more practical for patient use. For example, in one embodiment, the inflation conduit 48 may be coiled to condense the length and minimize tangling. The patient may pull on the inflation conduit 48, as needed, loosening the coil and extending the inflation conduit 48 to a desired length. After use, the inflation conduit 48 may contract back into a coil structure when released by the patient. The inflation conduit 48 may be manufactured from any suitable material, such as polyvinylchloride, silicone, rubber, or a combination thereof and coiled during or after extruding. In certain embodiments, the inflation conduit 48 may include a shape memory metal spring to maintain the integrity of the coiled structure. The shape memory spring may be overmolded so that it is integral to the inflation conduit 48. In certain embodiment, the inflation conduit 48 may also have a thick wall such that the ID small, reducing kinking of the conduit. For example, the inflation conduit 48 may have an ID of 2 mm or less.

In use, the syringe 160 may withdraw air from the balloon cuff 28 by pulling the plunger 164 outwardly and away from the distal end 162, as indicated by arrow 180. Once the distal end 168 of the plunger 164 abuts the stopping feature 172 the balloon cuff 28 is deflated and phonation is enabled as described above. The patient may return the withdrawn air from the balloon cuff 28 by pushing the plunger 164 toward the distal end 162 until the distal end 168 abuts the stopping feature 174, indicating the balloon cuff 28 is inflated and the patient's airway is sealed.

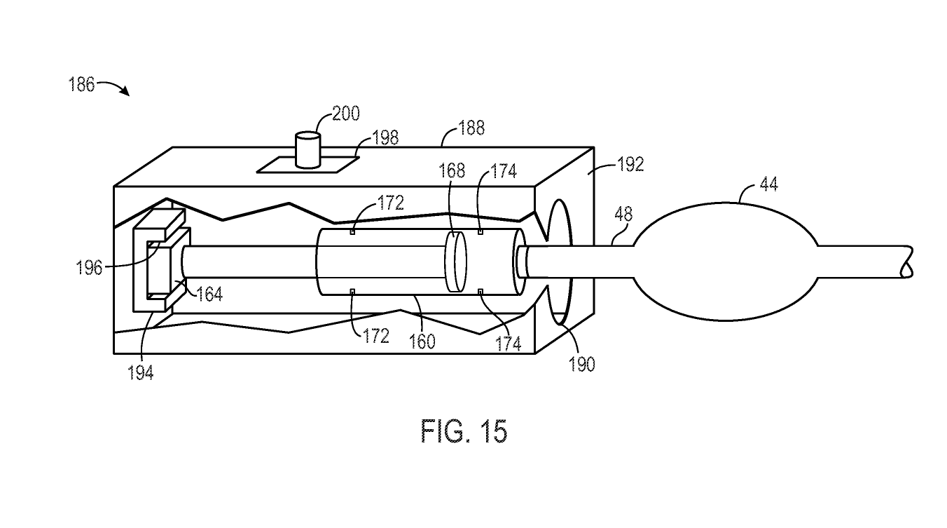

In other embodiments, a control system may be used to operate the syringe 160. FIG. 15 illustrates an embodiment of the syringe 160 in which the syringe 160 may be part of a syringe pump system 186. The syringe pump system 186 includes a housing 188 that houses the syringe 160. The housing 188 includes an opening 190 on a sidewall 192 such that the syringe 160 may be coupled to the PVT 44. The syringe pump system 186 may also include a driver head 194 that abuts a proximal end of the plunger 164. The driver head 194 may include a notch or recess 196 to attach the proximal end of the plunger 164 to the driver head 194. In use, a communications module 198 may be disposed on or inside the housing 188 of the syringe pump 186. The communications module 198 may be communicatively coupled, for example, through wireless techniques, to the driver head 194. In one example, the communications module 198 may include a switch or pushbutton 200 suitable for activating a deflation signal. Upon receipt of the signal, the syringe pump system 186 may deflate the balloon cuff 28 by, for example, activating the drive head 194 to pull the plunger 164 away from the distal end 162 as described above. In the same way, the pushbutton 200 may activate an inflation signal and activate the driver head 194 to push the plunger 164 towards the distal end 162 as described above. Accordingly, the patient may easily and discretely deflate and inflate the balloon cuff 28 as desired. In certain embodiment, the syringe pump system 186 may be battery operated.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims. Further, it should be understood that one or more components of the disclosed embodiments may be combined or exchanged with one another.

What is claimed is:

1. A tracheal tube system comprising:
a conduit;
an inflatable cuff disposed on the conduit and configured to seal a patient's airway;
an inflation line disposed along a portion of a length of the conduit and terminating in an opening within the inflation cuff; and
a speaking valve disposed at a proximal end of the conduit and in fluid communication with the inflatable cuff, wherein the speaking valve is fluidly coupled to the inflatable cuff via a one-way valve such that fluid from the cuff flows into the speaking valve when the one-way valve is open.

2. The system of claim 1, wherein the one-way valve is disposed in a part that protrudes from an exterior surface of the speaking valve.

3. The system of claim 1, comprising a second one-way valve disposed on a proximal end of the inflation line.

4. The system of claim 1, wherein the one-way valve is open during patient inhalation to deliver air from within the inflatable cuff into the patient's airway.

5. The system of claim 1, comprising an adapter having a first port and a second port, wherein the first port is coupled to the inflation line and the one-way valve and the second port is coupled to a suction source.

6. The system of claim 5, wherein the adapter is configured to provide selective fluid communication between the inflatable cuff and the speaking valve or the suction source.

7. The system of claim 1, wherein the one-way valve prevents re-inflation of the cuff.

8. The system of claim 1, wherein the system comprises at least one bellows configured to deliver air from the inflatable cuff to the speaking valve when compressed.

9. The system of claim 8, wherein a proximal end of the speaking valve comprises a cap configured to modulate a flow of air entering the patient's airway.

10. A speaking valve comprising:
a first one-way valve that opens during inspiration;
a second one-way valve fluidly coupled to an inflatable cuff on a tracheostomy tube, wherein the second one-way valve does not allow air to enter the inflatable cuff.

11. The valve of claim 10, comprising a port protruding from an exterior surface, wherein at least a portion of the second one-way valve is within the port.

12. The valve of claim 11, wherein the port is inserted into a proximal end of an inflation line of the tracheostomy tube.

13. The valve of claim 10, wherein the second one-way valve deflates the inflatable cuff during inspiration.

14. The valve of claim 10, comprising a cap on a proximal end of the speaking valve and configured to modulate a flow of air entering the speaking valve.

15. The valve of claim 14, wherein the cap is hinged.

16. The valve of claim 10, wherein the first one-way valve and the second one-way valve close during expiration.

17. The system of claim 10, wherein the first one-way valve and the second one-way valve direct air only into the tracheostomy tube.

18. The valve of claim 10, comprising an adapter disposed on a port that protrudes from an exterior surface of the speaking valve, wherein the adapter provides selective fluid communication between the inflatable cuff and the speaking valve or a suction source.

19. A tracheostomy kit comprising:
a speaking valve configured to be coupled to a tracheostomy tube and comprising a one-way valve configured to only deliver air into a patient's airway;
a first syringe comprising one or more features configured to provide a first fixed volume representative of a first cuff size; and
a second syringe comprising one or more features configured to provide a second fixed volume representative of a second cuff size.

20. The tracheostomy kit of claim 19, wherein the features comprise protrusions along an inner surface of the first syringe and the second syringe.

21. The tracheostomy kit of claim 19, wherein the speaking valve is configured to deflate an inflatable cuff of the tracheostomy tube.
